(19) United States
(12) Patent Application Publication
(10) Pub. No.: US 2004/0247676 A1
(43) Pub. Date: Dec. 9, 2004

(54) Use of multi-layer controlled-release tablet comprising ropinirole for the manufacture of medicament for the treatment of fibromyalgia

(76) Inventor: Gillian Frances Atkinson, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/492,890

(22) PCT Filed: Oct. 16, 2002
(86) PCT No.: PCT/EP02/11568

(30) Foreign Application Priority Data
Oct. 18, 2001 (GB) 0125088.5

(51) Int. Cl. A61K 9/24

(52) U.S. Cl. 424/472

(57) ABSTRACT

The invention relates to the use of a dosage formulation or tablet comprising a mixed matrix of hydrophilic and lipophilic components able to control the release rate of ropinirole or a pharmaceutically acceptable salt thereof from the formulation/tablet in the continual treatment of fibromyalgia.

The present invention relates to the use of a dosage formulation or tablet comprising a mixed matrix of hydrophilic and lipophilic components able to control the release rate of ropinirole or a pharmaceutically acceptable salt thereof from the formulation/tablet in the continual treatment of fibromyalgia.

Fibromyalgia is a common disabling disorder characterized by chronic musculoskeletal aches and pain, stiffness, general fatigue, and sleep abnormalities including diminished stage four sleep. Fibromyalgia is a chronic, painful disorder most commonly seen in general practice but also commonly seen in specialist rheumatology, neurology and pain practice and is often viewed as a musculoskeletal pain process. Examination of affected patients reveals increased tenderness at muscle and tendon insertion sites, known as “tender points”. Fibromyalgia patients experience severe morning stiffness and a generalized decrease of overall physical function, and they are often prone to headaches, memory and concentration problems, dizziness, numbness and tingling, and crampy abdominal or pelvic pain. Fibromyalgia affects 2-4% of the population and is most frequently found in women between 20 and 50 years old, though it can also affect men, the elderly and minors.

Diagnosis of fibromyalgia is often overlooked due to the general nature of the symptoms and the lack of objective diagnostic lab or x-ray abnormalities. The disorder is often concomitant with, masked by or confused with other diseases such as rheumatoid arthritis, chronic fatigue syndrome or irritable bowel syndrome. A physician can positively diagnose fibromyalgia syndrome by finding the symptoms of generalized musculoskeletal pain and pain at more than 11 of 18 defined characteristic “tender points” when finger pressure of about 4 kg is applied to the area. The total pain score for all 18 tender points is referred to as the “tender point index” of that patient. The efficacy of a particular fibromyalgia therapy is demonstrated by an observation of a statistically significant improvement in a patient's tender point index.

The etiology of fibromyalgia is not known but consideration has been given to genetic, traumatic, affective, and infectious processes as possibilities, or more likely to be a combination of these. Currently the best treatment available for fibromyalgia consists of a multidisciplinary approach combining analgesics, sleep aids, exercise programs emphasising stretching and cardiovascular fitness, relaxation techniques and other measures to reduce muscle tension, and educational and psychological support programs to reduce emotional and physical stress; the resulting benefits are usually disappointing. Numerous pharmaceutical regimes have been tried including treatment with serotonin modulators and antisera to endogenous psychoactive agents. Therapeutic response can be assessed by the reduction of pain in the tender point index and improvement in several generalized criteria such as physical function, stiffness, fatigue, depression, tenseness, etc. Responses to these various therapies have proven variable within a patient pool and have rarely exceeded modest relief of some symptoms.

In the past, there was a tendency to view fibromyalgia as a benign disorder which did not justify aggressive therapy which might carry with it any risk of adverse experience. However, that philosophy can no longer be justified considering the impact of this condition on the quality of life of affected individuals. Considering that the annual direct cost of fibromyalgia to the United States economy is estimated at $16 billion, there exists a significant need for more effective therapy for patients afflicted with fibromyalgia.

U.S. Pat. No. 6,277,875 describes the use of dopamine D2/D3 receptor agonists in the treatment of fibromyalgia, specifically providing evidence that pramipexole and ropinirole hydrochloride are particularly effective medicaments for such treatment. However, this patent discloses administration of an immediate release formulation containing ropinirole hydrochloride which for effective and continual relief is required to be administered in numerous doses during a 24 hour period. Furthermore, administration prior to sleep would provide immediate symptomatic relief but would not alleviate the symptoms of fibromyalgia for the entire duration of an average length of sleep (eg. 8 hours). In such a case, the patient would have disturbed sleep as the symptoms of fibromyalgia returned. Thus, there is a great need for a formulation containing ropinirole hydrochloride which is capable of sustained or controlled release over a 24 hour period so that administration of ropinirole is required only once per day, thereby ensuring optimal control of the symptoms of fibromyalgia.

According to a first aspect of the invention there is provided a use of a multi-layer controlled-release tablet comprising:

(a) one active layer containing: (i) ropinirole or a pharmaceutically acceptable salt thereof as an active substance, (ii) hydrophilic polymeric substances which swell and/or gel and/or erode upon contact with aqueous liquids, (iii) lipophilic substances, and (iv) adjuvant substances, wherein the weight ratio of the hydrophilic polymeric substances to the lipophilic substances contained in said active layer is in the range of 10:1 to 0.5:1; and

(b) one or more barrier layers containing one or more of: hydrophilic polymeric substances which swell and/or gel and/or erode upon contact with aqueous liquids, lipophilic substances, and adjuvant substances in the manufacture of a medicament useful in the treatment of fibromyalgia.

Multi-layer tablets prepared in accordance with the present invention are able to provide substantially equivalent (or identical) release kinetics for the same active substance when formulated at different amounts in the active substance containing layer in the multi-layer tablet. The pharmaceutical tablets of the invention have the advantage of releasing the carried ropinirole in a programmed way, preferably also avoiding the phenomenon of dose dumping, and therefore being able to meet specific therapeutic needs with the gradual and controlled release of ropinirole.

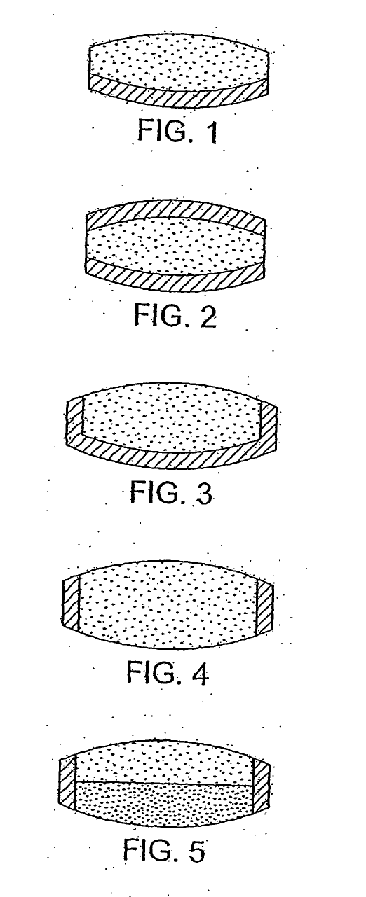

The multi-layer tablets of the present invention can be prepared as two layer tablets, three-layer tablets or greater numbers of layers if required. At least one layer will contain the ropinirole to be released from the tablet and at least one layer will be a barrier or support layer with respect to the ropinirole containing layer. Possible constructions of multi-layer tablets are shown in FIGS. 1 to 9. The tablets may have an overall substantially circular cross-section, or it may adopt a more oval cross-section or any other suitable geometric shape, for example rectilinear. The tablet may also be shaped as a caplet (capsule form tablet). As will be appreciated there are many potential arrangements of the layers in multi-layer tablets.

The layer containing the ropinirole can be referred to as the active layer. The layer that generally does not contain ropinirole can be referred to as a barrier layer or a support layer.

A simple two-layer tablet is shown in FIG. 1 where one lateral surface of the active substance containing layer (dotted) is covered by a barrier layer (hatched). A variation of this construction is shown in FIG. 2 where two barrier layers cover both exposed lateral surfaces of the active substance containing layer. In FIG. 3 a single barrier layer coats one lateral surface and the side of the active layer. The barrier layer is shown as being present in the form of an annular ring in FIG. 4 surrounding the active core and FIG. 5, an active core consisting of two active layers is shown surrounded by an annular ring of a barrier layer.

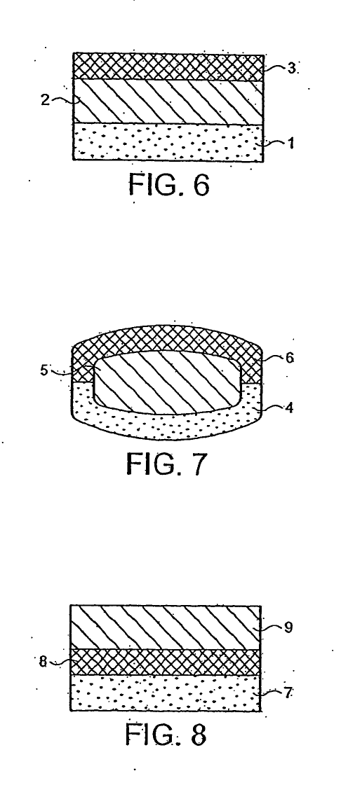

In FIG. 6 a three layer tablet is shown in which there is a first barrier layer (3) with an exposed upper lateral surface and sides, which is adjacent to a second active layer (2) with both lateral surfaces covered and the side of the layer exposed, which is in turn adjacent to a second active layer (1) where the bottom lateral surface is exposed and the side is exposed. The two active layers may contain different active substances or the same active substance in different amounts. FIG. 7 shows an alternative arrangement of the FIG. 6 embodiment, where the active substance layer (5) is wholly within the barrier layer (6) and the second active substance layer (4). In FIG. 8, a similar three-layer tablet is shown in which the barrier layer (8) is interposed between the two active substance containing layers (9) and (7).

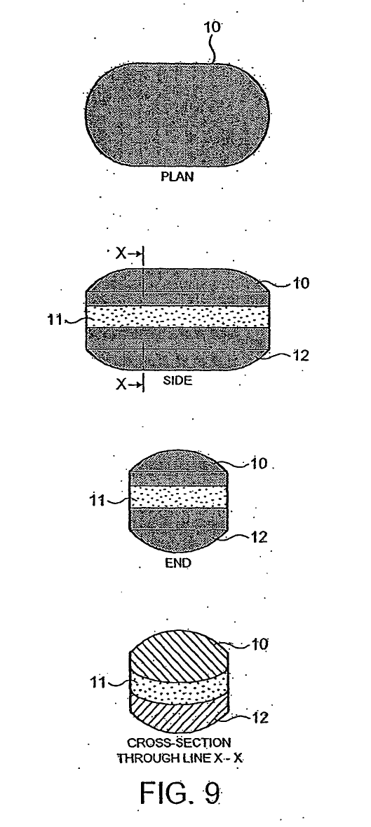

Another three layer tablet (caplet) construction is also shown in FIG. 9 in which the tablet has two external barrier layers (10, 12) and an active substance layer (11) interposed between the barrier layers.

In some tablet configurations, the barrier layer may also contain ropinirole such that it acts as a barrier layer with respect to a first active substance containing layer, but which itself is an active substance containing layer. Generally, in such embodiments, the ropinirole in the active layers is different in the separate layers, although arrangements in which ropinirole is present in the separate active layers in different amounts can also be envisaged.

The barrier layer(s) are meant to limit the release surface of the active layer, so as to allow that said carried ropinirole is released by the sole uncovered surface upon contact with the dissolution medium and/or the biological fluids according to kinetics that, in vitro, can be programmed according to precise methods, as will be highlighted in the given examples of the present invention.

Ropinirole, its chemical structure, processes for its preparation and therapeutic uses thereof, are more fully described in EP-A-0113964 (see Example 2), EP-A-0299602, EP-A-0300614, WO 91/16306, WO 92/00735 and WO 93/23035, and the contents of which are hereby incorporated by reference. “Ropinirole” as mentioned herein is defined as including pharmaceutically acceptable salts thereof. Most preferably, the ropinirole used in the tablet is in the form of the hydrochloride salt. Ropinirole is presently marketed as the HCl salt in an immediate-release tablet for the treatment of Parkinson's Disease (see also EP-A-0299602). Ropinirole can be synthesised by the advantageous method described in WO 91/16306.

The amount of ropinirole present, inclusive of pharmaceutically acceptable salts thereof, may be up to 12.0 mg, preferably from 0.75 mg to 12.0 mg, measured as the amount of ropinirole base present, that is excluding any amount of acid (for example, hydrochloric acid, HCI) added to form any ropinirole salts. The amount of ropinirole present, inclusive of pharmaceutically acceptable salts thereof, may be up to 12.0 mg, preferably from 0.75 mg to 12.0 mg, measured as the amount of ropinirole base present, per 150 mg of active layer present. See Examples 1 to 6 hereinafter.

Ropinirole is preferably contained in a percentage between 0.05% to 50% by weight of the active layer, more preferred ranges of ropinirole are 0.05% to 40%, 0.05% to 30%, 0.05% to 10%, 0.05% to 20%.

Natural or synthetic hydrophilic polymeric substances, can be used in the preparation of said active layer which are biocompatible and/or biodegradable materials and pharmaceutically acceptable, e.g. polyvinylpyrrolidone in particular non-cross-linked polyvinylpyrrolidone (e.g. of molecular weight 30,000-400,000), hydroxypropylcellulose with a molecular weight of from 100,000 to 4,000,000, sodium carboxymethylcellulose (e.g. non-cross-linked, e.g. typical molecular weight 90,000-700,000), carboxymethylstarch, potassium methacrylate-divinylbenzene copolymer, hydroxypropylmethylcellulose with a molecular weight between 2,000 and 4,000,000, polyethyleneglycols of different molecular weight preferably between 200 and 15,000 (more preferably 1000-15000) and polyoxyethylenes of molecular weight up to 20,000,000 (more preferably 400,000-7,000,000), carboxyvinylpolymers, poloxamers (polyoxyethylene-polyoxypropylene copolymer), polyvinylalcohols, glucanes (glucans), carrageenans, scleroglucanes (scleroglucans), mannans, galactomannans, gellans, xanthans, alginic acid and derivatives (e.g. sodium or calcium alginate, propylene glycol alginate), polyaminoacids (e.g. gelatin), methyl vinyl ether/maleic anhydride copolymer, carboxymethylcellulose and derivatives (e.g. calcium carboxymethylcellulose), ethylcellulose, methylcellulose, starch and starch derivatives, alpha, beta or gamma cyclodextrin, and dextrin derivatives (e.g. dextrin) in general. The hydrophilic polymeric substance is therefore one which can be described as a controlled release polymer or a polymeric substance which is capable of achieving controlled release (CR).

More preferably for achieving advantageous controlled release of ropinirole the hydrophilic polymeric substances in the active layer comprise one or more of the following: hydroxypropylcellulose with a molecular weight of from 100,000 to 4,000,000, hydroxypropylmethylcellulose (HPMC) with a molecular weight between 2,000 and 4,000,000 (more preferably between 10,000 and 1,500,000 molecular weight, still more preferably between 20,000 and 500,000 molecular weight, most preferably about 250,000 molecular weight), ethylcellulose or methylcellulose. The most preferred controlled release polymer is HPMC.

Hydrophilic polymeric substances such as sodium carboxymethylcellulose and/or calcium carboxymethylcellulose that act as viscosity-increasing agents/polymers or “cage-forming” components are also preferred components e.g. of the active layer. The provision of these viscosity-increasing polymers in the active layer is preferred because these help to reduce the “dose-dumping” effects occasionally seen with ropinirole whereby a significant minority of ropinirole can be released from the active layer in the first (say) hour after oral administration. Thus, it is preferred for this purpose that the hydrophilic polymeric substances in the active layer comprise sodium carboxymethylcellulose, carboxymethylcellulose or a derivative (e.g. calcium carboxymethylcellulose), hydroxypropylcellulose with a molecular weight of from 100,000 to 4,000,000, a carboxyvinylpolymer, a carrageenan, a xanthan, alginic acid or a derivative (e.g. sodium or calcium alginate, propylene glycol alginate), ethylcellulose, methylcellulose, dextrin and/or maltodextrin. Most preferred for this purpose is sodium carboxymethylcellulose (NaCMC) (e.g. non-cross-linked, e.g. typical molecular weight 90,000-700,000). The present invention also comprehends the use of other equivalent polymers able to act as viscosity-increasing agents and/or “cage-forming” components.

It is more preferred that the hydrophilic polymeric substances in the active layer comprise both the above-mentioned preferred controlled release polymers and the above-defined viscosity-increasing polymers. Thus it is preferred that the hydrophilic polymeric substances in the active layer comprise:

(a) one or more of the following: hydroxypropylcellulose with a molecular weight of from 100,000 to 4,000,000, hydroxypropylmethylcellulose (HPMC) with a molecular weight between 2,000 and 4,000,000, ethylcellulose or methylcellulose; and

(b) sodium carboxymethylcellulose, carboxymethylcellulose or derivatives (e.g. calcium carboxymethylcellulose), hydroxypropylcellulose with a molecular weight of from 100,000 to 4,000,000, a carboxyvinylpolymer, a carrageenan, a xanthan, alginic acid or a derivative (e.g. sodium or calcium alginate, propylene glycol alginate), ethylcellulose, methylcellulose, dextrin and/or maltodextrin.

Thus, while the controlled release polymer (a) such as HPMC is still swelling and/or gelling gradually in the first hour-or-so after oral administration of the tablet, when it may be less effective in controlling release of soluble active substances such as ropinirole from the active layer, the viscosity-increasing polymer (b) such as sodium carboxymethylcellulose (NaCMC) reduces the release of ropinirole from the active layer. Without being bound by theory, ionic viscosity-enhancers like NaCMC might also to interact with the hydroxypropyl groups of, for example, HPMC to boost synergistically the hydration and swelling rate of HPMC leading to greater gel strength.

Thus, the most preferred combination is that the hydrophilic polymeric substances in the active layer include (or are) HMPC and sodium carboxymethylcellulose.

Preferably, the hydrophilic polymeric substances have an HLB value of at least 10 (see A. Gennaro and J. Remington, Remington's Pharmaceutical Sciences, 18^{th} edition, Mack Publishing Company, Easton, Pa., 304 (1990) and W. C. Griffin, J. Soc. Cosmetic Chemists, vol. 1, page 311, 1949 for HLB values and measurement thereof). Said hydrophilic polymeric substances make up between 1% and 75% of the weight of the active layer, but preferably are present in a percentage between 5% and 65% and/or between 30 and 75%, more preferably 43-75% or 43-67% or 43-65%. Any HPMC present in the active layer is preferably present in about 40-63% by weight of the active layer. The viscosity-increasing polymers mentioned above, such as sodium carboxymethylcellulose, if present are preferably present in up to 20% by weight of the active layer, more preferably (especially for NaCMC) 3-20%, 5-20%, 7-15%, or about 10% by weight of the active layer.

For all the polymers cited different types are commercially available characterised by different chemical, physical, solubility and gelification properties. In particular, as regards, hydroxypropylmethylcellulose various types with a different molecular weight (between 1,000 and 4,000,000, preferably from 2,000 to 4,000,000, even more preferably between 10,000 and 1,500,000 molecular weight, still more preferably between 20,000 and 500,000 molecular weight, most preferably about 250,000 molecular weight) can be used and with different degrees of substitution. Said types of hydroxypropylmethylcellulose have differentiated characteristics being mainly erodible or able to be gelled, depending on the viscosity and the degrees of substitution (D.S.) present in the polymeric chain. Gellable HPMCs (e.g. Methocel K grades) are preferable to erodible HPMCs (e.g Methocel E grades). The polyethyleneglycols and polyoxyethylenes show identical behaviour: in fact, different hydrophilic and gelification properties correspond to different molecular weights.

The molecular weight of polymers and the 2% viscosity of polymers can be directly correlated (“METHOCEL™ in Aqueous Systems for Tablet Coating”, page 12, published by The Dow Chemical Company—www.dow.com—METHOCEL™ is a trademark of The Dow Chemical Company) where viscosity of a polymer is defined as viscosity of a 2% aqueous solution at 20° C. measured as mPa.seconds. Viscosity is measured in Pascal seconds (SI units) or in poise (c.g.s. units), where 1 centipoise=10⁻³ Pa.sec. So for example, METHOCEL™ K100M has an approximate molecular weight of 246,000 and a corresponding 2% viscosity of 100,000 mPa.sec (based on an average viscosity of 80,000 to 120,000 mPa.sec.); METHOCEL™ K4M has an approximate molecular weight of 86,000 and a corresponding 2% viscosity of 4,000 mPa.sec; and METHOCEL™ K100LV has an approximate molecular weight of 27,000 and a corresponding 2% viscosity of 100 mPa.sec. For this reason, the preferred molecular weight ranges of the polymeric substances, for example the hydroxypropylmethylcellulose polymers can also be defined in terms of viscosity.

One preferred viscosity range for the hydroxypropylmethylcellulose polymers as defined above may be in the range of from 50 to 150,000 mPa.sec, suitably 80,000 to 120,000 mPa.sec (e.g. K100M, as in the active and barrier layers of Examples 1-9). This applies both to the active layer (discussed above) or the barrier/support layer(s) (discussed below).

In an alternative embodiment, in order to obtain a faster release rate, the viscosity range for the hydroxypropylmethylcellulose polymers in the active and/or barrier layer(s) may be in the range of from 50 to 25,000 mPa.sec (including Methocels K4M, K15M, K100LV). In this embodiment, preferably some or all of the HPMC polymers have a viscosity in the range of from 1000 to 25,000 mPa.sec (including Methocels K4M & K15M but not K100LV or K100M). More preferably, HPMC polymers having a viscosity in the range of from 1000 to 25,000 mPa.sec are present in the active or barrier layer in a percentage of from 5 to 50% by weight of the active or barrier layer. In particular, Examples 10 and 11 hereinafter have 10% and 40 weight % respectively of such HPMC (K4M) in their barrier layers which gives a slightly faster release profile in vitro than the ca. 45 wt % K100M HPMC present in the barrier layers of Examples 1-6 and 7-9, as inter alia ropinirole migrates faster through the barrier layers. Preferably, the proportion of low-viscosity HPMCs having from 50 to <1000 mPa.sec viscosity (including Methocel K100LV) contained in the active or barrier layers is less than 30% by weight of that layer—e.g. Example 10 has 20 wt % of such HPMC (K100LV) as well as 10 wt % K4M HPMC in the barrier layer. Up to 30% low-viscosity HPMC in active or barrier layer can increase water uptake and aid gelling, increasing the matrix viscosity and decreasing the release rate, but greater amounts are not preferred.

In an alternative embodiment of the invention, there is provided a tablet as previously defined, in which the active layer contains polymeric material with slow swelling and/or gellification and/or erosion and/or solubility properties.

A fundamental characteristic of the tablets of the present invention is that, for the formulation both of the layer containing ropinirole and the barrier layers, lipophilic substances are utilised, for example natural fats (coconut, soya, cocoa) as such or totally or partially hydrogenated, beeswax, polyethoxylated beeswax, mono-, bi- and tri-substituted glycerides, glyceryl palmitostearate, glyceryl behenate (glyceryl tribehenate C₆₉H₁₃₄CO₆, e.g. Compritrol 888, where behenic acid=docosanoic acid=C₂₁H₄₃COOH), diethyleneglycol palmitostearate, polyethyleneglycol stearate, polyethyleneglycol palmitostearate, polyoxyethylene-glycol palmitostearate, glyceryl monopalmitostearate, cetyl palmitate, mono- or di-glyceryl behenate (glyceryl mono-behenate or glyceryl di-behenate), fatty alcohols associated with polyethoxylate fatty alcohols, cetyl alcohol, stearic acid, saturated or unsaturated fatty acids and their hydrogenated derivatives, hydrogenated castor oil and lipophilic substances in general. In certain preferred embodiments of the invention, the lipophilic substances are selected from hydrogenated castor oil and glyceryl behenate.

Preferably, the lipophilic substances have an HLB value of less than 10, more preferably, less than 5.

Preferably, the lipophilic substances make up between 1% and 70% of the active layer weight, but preferably are present in a percentage between 5% and 55%, more preferably 5-35%.

The weight ratio between the content of hydrophilic polymeric substances and lipophilic substances, in the layer containing ropinirole, is between 10:1 and 0.5:1 (i.e. in the range of 10:1 to 0.5:1), suitably between 10:1 and 1:1 (i.e. in the range of 10:1 to 1:1), but preferably between 7:1 and 1:1 (i.e. in the range of 7:1 to 1:1).

Besides the previously cited hydrophilic polymers and the lipophilic substances, lipophilic and/or substances of amphiphilic nature may be used in the formulation, in which the hydrophilic portion can be represented by glycerol molecules or other polyalcohols or polyethyleneglycol molecules (PEG) of molecular weight between 100 and 10,000, whereas the lipophilic part is represented by unsaturated and/or saturated fatty acids, in hydrogenated vegetable oil form. The association of the hydrophilic portion with the lipidic chain is obtained by esterification reactions or partial alcoholysis of hydrogenated vegetable oils by PEG molecules or glycerol or other polyol. In this way compounds characterised by a different degree of hydrophilicity are obtained that can be assessed by measuring the Hydrophilic-Lipophilic Balance (HLB). Triglycerides are available with an HLB value between 1 and 2, diglycerides with HLB between 2 and 3, monoglycerides with HLB between 3 and 4, PEG diesters with HLB between 6 and 15, PEG monoesters with HLB between 10 and 17. In practice, increasing the HLB values increases the hydrophilic tendency and, obviously, decreases the lipophilic tendency. Tablets according to the present invention may therefore also contain polymeric substances of a lipophilic nature.

Finally, adjuvants normally used in the pharmaceutical technique may be employed, for example, diluents, binders, lubricants, glidants and non-stick types, for example, starch, mannitol, lactose, sorbitol, xylitol, talc, stearic acid, sodium benzoate, magnesium stearate, colloidal silica, maltodextrin, and other excipients known to the expert in the field.

In order to promote the penetration of water and/or aqueous fluids in the layer or nucleus, hydrophilic diluents, for example, mannitol, lactose, starches of different origins, sorbitol, xylitol are introduced, or substances with wetting properties and/or those generally encouraging the penetration of water in the solid are preferably carried in the formulation.

Moreover, diluents, binders, lubricants, buffers, non-stick substances, glidants and plasticising substances can be employed as well as others capable of giving said layer the desired characteristic as will be better illustrated in the examples quoted later on.

Said adjuvants are preferably contained in a percentage between 5% to 50%, preferably from 10% to 40% or 20 to 50% or 20% to 35% of the weight of said active layer. The weight ratio of ropinirole (i) to that of the adjuvant component (iv) in the active layer can be in the range of from 0.001:1 and 4:1, suitably of from 0.003:1 to 3:1.

The polymeric substances employed for the preparation of the barrier layer in association with other adjuvants, are able to provide a barrier (applied by compression) which proves impermeable to the carried ropinirole in the underlying layer for a time period that strictly depends on its composition, which can vary from 1 hour to approx. 20-24 hours or more. In such case, the release of ropinirole in the stated periods (e.g. during the first hour after oral administration/immersion in aqueous fluids) occurs only from the surface of the tablet not covered by the barrier. “Impermeability” is to be construed accordingly. Preferably, during the first hour after oral administration or immersion in aqueous liquids (e.g. water), release of ropinirole occurs substantially only from the surface of the tablet not covered by the barrier.

To test the impermeability of the barrier layer to the release of ropinirole, various suitable tests can be conceived by persons skilled in the art of pharmaceutical tablet formulation. However, one such test may be based on selective coating of the free surfaces of the active layer with a suitable substance, such as an enteric coat (for example, “Eudragit”), or a waxy material (for example, beeswax) such that normal release of ropinirole does not occur through these surfaces.

An in vitro dissolution test can then be performed in which the dissolution fluid can be sampled at appropriate time points. In this way, the point in time can be determined at which ropinirole is released through the barrier layer through the interaction of the component substances of the barrier layer with the aqueous environment (that is to say the point in time at which the polymers of the barrier layer permit release). Alternatively, the free surfaces of the barrier layer(s) can be selectively coated as above and a dissolution test performed. The release profile obtained would correspond to that of an uncoated tablet up to the point in time at which ropinirole was able to permeate the barrier layer and be released from the uncoated tablet.

As noted above, the barrier layer may be superposed over one or more free surfaces of the active layer in the tablet. Generally, the barrier layer will form a layer to cover one or more lateral surfaces of the active layer. In a preferred embodiment of the present invention, tablets are provided in which one or more barrier layers are used to cover one or both surfaces or bases of the active layer. Such arrangements therefore provide for a bi-layer or a tri-layer tablet.

The natural synthetic hydrophilic polymeric materials, usable in the formulation of the barrier layer, can be chosen from among those listed for the preparation of the active layer. Said polymeric substances can be present in a percentage of 5 to 90%, with respect to the total weight of said layer but, preferably, between 25% and 85%.

Said polymeric substances, utilised individually or mixed together and mixed with the lipophilic substances, are able to bring about the impermeability in the release of the carried ropinirole in the underlying layer for a time interval that can vary from 1 hours to approx. 20-24 hours or more, depending on the composition.

For the preparation of the barrier layer such lipophilic substances can be chosen from those listed for preparation of the active layer. Said lipophilic substances can be present in a percentage between 5% and 70% with respect to the total weight of said layer but, preferably, between 5% and 55%.

The weight ratio of hydrophobic swelling and/or gelling and/or erodible polymeric substances to lipophilic substances contained in the barrier layer can be in the range of 1:1 and 7.5:1, suitably of from 1.5:1 to 4:1, and preferably from 2:1 to 3.5:1.

Said barrier layer(s), applied by compression, can have a thickness between 0.1 and 4.5 mm. The matrix preparation can be carried out by the compression of powder or granular mixtures, for example by blending followed by dry compression or wet granulation followed by compression, and preferably working between 1000 and 5000 Kg/cm².

In general, tabletting can be through direct compression. i.e. a mixture of dry powders being compressed, but this can sometimes cause quality issues such as segregation, poor flow etc. These issues can be improved by the use of granulation techniques on all or part of the constituent mix.

Granulation is a process in which powder particles are agglomerated together to form granules. This can be carried out to:

1. improve the flow properties of a powder mixture,

2. prevent segregation of the constituent powders (improve homogeneity),

3. improve compression characteristics,

4. achieve densification of powder mixes, and/or

5. achieve alteration of particle size/shape/hydrophilic properties

The tablet of the invention may be prepared by dry granulation. Dry Granulation is granulation by compression of powders by either slugging or roller compaction. It is essentially a densification process.

Slugging is where a crude compact (slug) is produced to a set weight/thickness for a given diameter of slug. These slugs are then reduced by either grating or commuting mill to produce granules of the required particle size/range.

Roller compaction or Chilsonating is where a powder mix is forced via an auger between 2 rollers (which can be smooth or grooved). Compaction of this material is controlled by the feed rate to the rollers and the hydraulic force of the rollers being pushed together. The resulting compact (called a ribbon or strip) is then reduced by either grating or commuting mill to produce granules of the required particle size/range.

Where dry granulation is used, the adjuvants often differ slightly compared to wet granulation. For example, instead of lactose monohydrate (often used in wet granulation), one preferably uses spray-dried lactose preferably containing amorphous lactose (e.g. Fast-Flo lactose, Seppic, Paris, France).

However, the tablet of the invention is preferably prepared by wet granulation. Wet Granulation is the most widely used granulation technique, and involves powder densification and/or agglomeration by the incorporation of a granulation fluid/medium to the powder mix. Wet granulation can be aqueous-based or solvent-based, e.g. based on organic solvents. Shear is dependent on the speed of the granulator paddle/blade through the powder. Various mixer designs are available, for example:

Wet High Shear, (rotating high shear forces (Fielder))

Wet Low Shear, (rotating low shear forces (Planetary mixer))

Wet Low Shear Tumble, (spraying in to tumble mixer with/without intensifier bar)

Extrusion, (Wet solids pushed through classified screen)

Rotary Granulators, (Spheronisation, Marumerisation—spinning disk or walls of a vessel)

Spray granulation in a fluidised Bed, or

Spray dry granulation.

For the formulation of said layer-barrier, which can be applied by compression, possible adjuvants, in particular diluents, include those traditionally used in the preparation of solid forms. For example magnesium stearate, stearic acid, sodium stearate, talc, sodium benzoate, boric acid, polyethylene glycols and/or colloidal silica can be employed.

In addition diluent, lubricating, non-stick and glidant substances and other substances may be used capable of giving said layer the desired characteristic, as will be better illustrated in the examples quoted later on. Other possible components include substances able to impart a colour to the eventual tablet layer prepared and formulated in the multi-layer tablet, for example iron oxide (yellow ferric oxide).

In addition a covering could be applied to said finished tablets by a coating process and/or any other process well known to experts in the field. An example of a coating is “OPADRY OY-S-28876 WHITE”. OPADRY OY-S-28876 WHITE is 63% HPMC 2910 6cP, 7% PEG 400, 30% TiO2. Red/pink (0.01-0.25%) and/or yellow (0.1 to 1.5%) colourings can also be added (iron oxides), the HPMC varying between 61-66%. An alternative blue coating uses 31-32% each of HPMC 2910 3cP and HPMC 2910 5cP, 8% PEG400, 23-24% TiO2, 1% polysorbate, and indigotine as blue dye at 45%.

A colourant layer or a film of gastroresistant and enterosoluble polymeric material may also be applied to said finished tablets, so as to allow the activation of the system only after the tablet has reached the duodeno-intestinal tract. Pharmaceutical systems of the latter type can be utilised for the accomplishment of tablets specifically designed to release ropinirole in the latter part of the intestinal tract i.e. at colon level. In order to attain gastroresistance, polymeric materials such as cellulose acetophthalate, cellulose acetopropionate, cellulose trimellitate, polymers and acrylic and methacrylic copolymers can be used of different molecular weights and with solubility which depends on different pH values. Said materials can be applied to the finished pharmaceutical form (active layer and the barrier layer(s)) by the classical coating process, utilising solutions in organic solvents or aqueous dispersions and spraying or fluidised bed nebulisation. Said gastro-resistant and enterosoluble materials can likewise be utilised in association with retarder polymers.

One innovative embodiment is characterised in that is possible to achieve the claimed therapeutic system by utilising the production technologies currently in use, i.e. the system may be immediately set up at industrial level.

One preferred embodiment of a tablet of the present invention comprises a tablet as previously described in which the active layer consists of components (i) to (iv) wherein ropinirole is present in a weight percentage of 0.05% to 20% by weight of the active layer, the adjuvant substances are present in a weight percentage of 5% to 50% by weight of the active layer, and the weight ratio of the hydrophilic polymeric substances to the lipophilic substances is in the range of from 7:1 to 1:1.

Alternatively, the active layer consists essentially of components (i) to (iv) wherein ropinirole is present in a weight percentage of 0.05% to 20% by weight of the active layer, the adjuvant substances are present in a weight percentage of 5% to 50% by weight of the active layer, and the weight ratio of the hydrophilic polymeric substances to the lipophilic substances is in the range of from 7:1 to 1:1.

In certain preferred embodiments, the hydrophilic polymeric substance may comprise hydroxypropylmethylcellulose of molecular weight 2,000 to 4,000,000, sodium carboxymethylcellulose or calcium carboxymethylcellulose.

The tablet may be characterised by comprising (i) ropinirole present in a weight percentage of 0.05% to 20% by weight of the active layer, (ii) the hydrophilic polymeric substance being hydroxypropylmethylcellulose, sodium carboxymethylcellulose or calcium carboxymethylcellulose. (iii) the lipophilic substance being hydrogenated castor oil or glyceryl behenate, and (iv) the adjuvant substances being present in a weight percentage of 5% to 50% by weight of the active layer, in which the weight ratio of the hydrophilic polymeric substances to the lipophilic substances being in the range of from 7:1 to 1:1.

According to the use of the present invention, one or more multi-layer tablets can be administered once per day to the human need of treatment of fibromyalgia, or a single multi-layer tablet can be administered once per day. The controlled-release ropinirole tablet of the present invention is expected to be advantageous compared to the marketed ropinirole immediate-release (IR) formulation because it should allow a more constant and/or lower systemic concentration/Cₘₐₓ over a 24-hr period, avoids the necessity with the IR tablet of taking ropinirole three times a day, and should avoid some of the side-effects which are possible when IR ropinirole is administered. See especially the advantageous approx. 24 hour in vitro release shown inter alia in ropinirole Examples 1-6 hereinafter.

It will be appreciated that the amount of ropinirole used within the multi-layer controlled-release tablet according to the present invention will be such to result in the clinically determinable improvement in or suppression of symptoms of fibromyalgia, such as musculoskeletal pain. An improvement in such symptoms includes both a reduction in intensity and frequency of musculoskeletal pain and a complete cessation of musculoskeletal pain for a sustained period. Typically effective amounts of ropinirole will generally range from about 0.1 mg/day to about 50 mg/day, more preferably about 1 mg/day to about 40 mg/day and most preferably about 2 mg/day to about 30 mg/day.

Patients are initially treated with ropinirole at the low end of the recommended dose, for example a dose of about 1 mg once per day. An example of a typical dose regime for treatment of fibromyalgia with ropinirole may then involve increasing the amount of ropinirole gradually on a weekly basis until the patient exhibits an therapeutic effect or intolerance. Table 1 details 2 suitable examples of such a dosing regime. Alternatively, if desired, a more rapid dosage regime may also be used.

TABLE 1

| | |
|---|---|
| An Example of a | An Example of a |
| Standard | Rapid |
| Dosing Regime | Dosing Regime |

| | Dose (mg once | | Dose (mg once |
|---|---|---|---|
| Week | per day) | Week | per day) |
| 1 | 1 | 1 | 2 |
| 2 | 2 | 2 | 4 |
| 3 | 3 | 3 | 6 |
| 4 | 4 | 4 | 8 |
| 5 | 6 | 5 | 12 |
| 6 | 8 | 6 | 16 |
| 7 | 10 | 7 | 20 |
| 8 | 12 | 8 | 24 |
| 9 | 14 | 9 | 28 |
| 10 | 16 | 10 | 32 |
| 11 | 20 | 11 | 36 |
| 12 | 24 | 12 | 40 |
| 13 | 28 | | |
| 14 | 32 | | |
| 15 | 36 | | |
| 16 | 40 | | |

The effective dose of ropinirole is usually between about 1 mg per day to about 50 mg per day. More usually, the effective dose is between about 3 mg and about 30 mg per day.

It will be understood, however, that the specific dose level for any particular patient will depend upon a variety of factors including the age, body weight, general health, sex, diet, time of administration, route of administration, rate of excretion, drug combination, and the severity of fibromyalgia. In general, the dosage of ropinirole should be increased gradually from a starting dose of about 1-2 mg of ropinirole per day and then increased every 1-7 days to a maximum dose of per day of about 30.0 mg of ropinirole per day. Providing patients do not experience intolerable side effects, the dosage should be titrated to achieve a maximal therapeutic effect.

The invention also provides a method of treatment of fibromyalgia which comprises administration of a multi-layer controlled-release tablet as herein defined.

Preferred features for the second and subsequent aspects of the invention are as for the first aspect mutatis mutandis.

The invention will now be further described by way of reference to the following Examples and Figures which are provided for the purposes of illustration only and are not to be construed as being limiting on the invention. Reference is made to a number of Figures in which:

FIG. 1 shows a transverse section through a bilayer tablet in which the barrier layer is shown by hatching and the active substance containing layer by dots.

FIG. 2 shows a transverse section through a trilayer tablet which has upper and lower barrier layers and a central active layer

FIG. 3 shows a transverse section through a two-layer tablet, in which the barrier layer coats a lateral surface and the side of the active layer.

FIG. 4 shows a transverse section through a two-layer tablet in which the barrier layer is present as an annular ring around the active core.

FIG. 5 shows a transverse section through a tablet of FIG. 4 in which the active core consists of two different active layers.

FIG. 6 shows a transverse section through a trilayer tablet in which the barrier layer (3) is superposed on active layer (2), which in turn is superposed on active layer (1).

FIG. 7 shows a transverse section through a trilayer tablet in which the first active layer (5) is contained within barrier layer (6) and a second active layer (4).

FIG. 8 shows a transverse section through a trilayer tablet in which the barrier layer (8) is present interposed between active layers (9) and (7).

FIG. 9 shows plan, side elevation and end elevation views of a three layer caplet, in which an active substance layer (11) is interposed between barrier layers (10,12); a cross-sectional view is shown through line X-X.

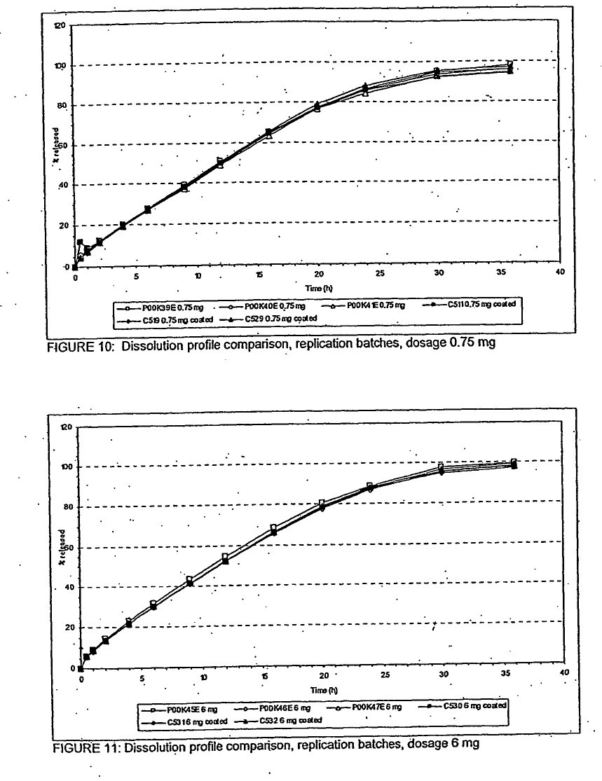

FIG. 10 shows a dissolution profiles of comparison, replication batches at dosages of 0.75 mg ropinirole measured as effective free base to investigate the influence of coating on release—results shown for tablet P00K39E shown as “□”, tablet P00K40E shown as “⋄”, tablet P00K41E shown as “Δ”, tablet C511 shown as “▪”, tablet C519 shown as “♦”, and tablet C529 shown as “▴”. Results shown as percentage drug released (%) over time (hours).

FIG. 11 shows a dissolution profiles of comparison, replication batches at dosages of 6 mg ropinirole measured as effective free base to investigate the influence of coating on release—results shown for tablet P00K45E shown as “□”, tablet P00K46E shown as “♦”, tablet P00K47E shown as “Δ”, tablet C530 shown as “▪”, tablet C531 shown as “♦”, and tablet C532 shown as “▴”. Results shown as percentage drug released (%) over time (hours).

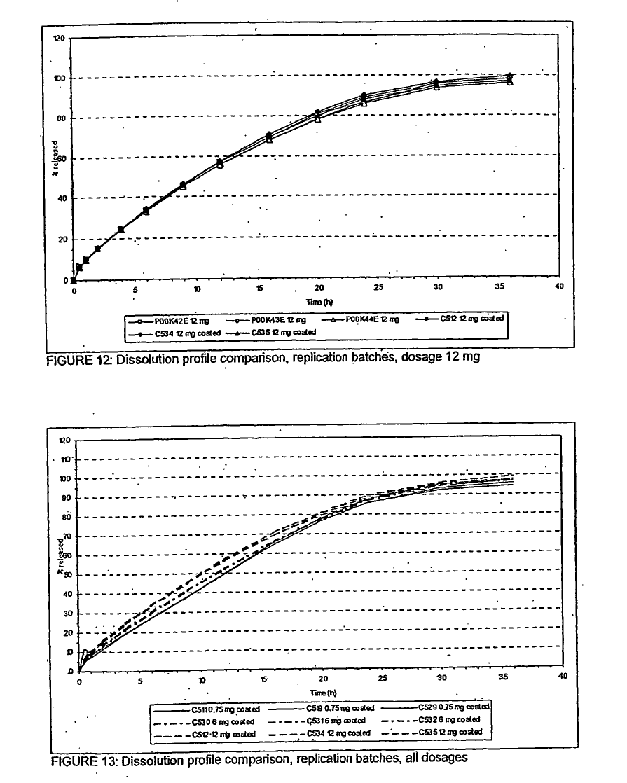

FIG. 12 shows a dissolution profiles of comparison, replication batches at dosages of 12 mg ropinirole measured as effective free base to investigate the influence of coating on release—results shown for tablet P00K42E shown as “□”, tablet P00K43E shown as “⋄”, tablet P00K44E shown as “Δ”, tablet C512 shown as “▪”, tablet C534 shown as “♦”, and tablet C535 shown as “▴”. Results shown as percentage drug released (%) over time (hours).

FIG. 13 shows dosage influence on coated tablet displayed as a comparison of dissolution profiles of ropinirole at dosages of 0.75 mg, 6 mg and 12 mg measured as free base (results of replication batches). Results shown as percentage drug released (%) over time (hours), where tablets C511, C519 and C529 are 0.75 mg ropinirole shown as “—”, where tablets C530, C531 and C532 are 6 mg ropinirole shown as “-—-—”, and where tablets C512, C534 and C535 are 12 mg ropinirole shown as “------”.

### EXAMPLE 1

### Systems Consisting of a Single Three-Layer Tablet—0.75 mg Ropinirole

In Example 1, the first layer consists of 130 mg of a “barrier” layer, the second layer (slow release) contains 0.86 mg of Ropinirole HCl equal to 0.75 mg of base; the third layer consists of 120 mg of a “barrier” layer.

1(a) Preparation of the Granulate Utilised for the Preparation of the Slow Release Base Layer Containing 0.86 mg of Ropinirole HCl Equal to 0.75 mg of Base.

| Component | Amount (mg) |
|---|---|
| Ropinirole HCl equal to 0.75 mg of base | 0.86 mg |
| Hydroxypropylmethylcellulose (HPMC) | 61.50 mg (41 wt %) |
| (Methocel ® K 100 M, | |
| Colorcon, Orpington, UK) | |
| Sodium carboxymethylcellulose (NaCMC) | 15.00 mg (10 wt %) |
| (Blanose 9 M31XF) | |
| Maltodextrin NF (Lycatab DSH) | 7.50 mg (5 wt %) |
| Lactose (C. Erba, Milan, I) | 47.74 mg |
| Hydrogenated castor oil (Cutina HR-Henkel, D) | 15.00 mg (10 wt %) |
| Magnesium stearate (C. Erba, Milan, I) | 1.50 mg (1 wt %) |
| Colloidal silica (Syloid 244, Grace GmbH, | 0.90 mg |
| Worms, D) | |
| Total | 150.00 mg |

In total, the active layer contains 51 weight % of the gellable, swellable and/or erodible hydrophilic polymers (HPMC+NaCMC), or 56 wt % if one includes maltodextrin.

Mix the Ropinirole and a part of the lactose for 20 minutes in a suitable mixer-granulator, (type Niro-Fielder PMA). Add the hydroxypropylmethylcellulose, sodium carboxymethylcellulose, hydrogenated castor oil, maltodextrin and the remainder of the lactose and mix for 10 minutes, wet with water (approx. 30% of the weight of the products utilised). The granulate obtained is desiccated in a fluid-bed desiccator (type Niro-Fielder TSG 2) until constant weight is reached. Pass over an 0.800 mm mesh sieve oscillating granulator again. Add the silica and mix in a cubic mixer for 20 minutes, then add the magnesium stearate and mix for a further 10 minutes. In this way a granulate (granulate 13(a)) is obtained with good slide and compaction properties. The granulate is subjected to the compression stage as described later on.

1(b) Preparation of the Granulate Making Up the Barrier-Layers.

| Component | Amount |
|---|---|
| Hydroxypropylmethylcellulose | 44.76% |
| (Methocel ® K 100 M, Colorcon, | (or 44.75%) |
| Orpington, UK) | |
| Mannitol (C. Erba) | 23.60% |
| Glyceryl behenate (Compritol 888 | 25.00% |
| Gattefosse, St. Priest; F) | |
| Polyvinylpyrrolidone (PVP) (Plasdone ® | 5.00% |
| K29-32, I.S.P.) | |
| Yellow iron oxide FCF (Sicovit Gelb 10-BASF | 0.24% |
| Koln; D) | (or 0.25%) |
| Magnesium stearate (USP grade, C. Erba, | 1.00% |
| Milan, I) | |
| Colloidal silica (Syloid 244, Grace GmbH, | 0.40% |
| Worms, D) | |
| Total | 100.00% |

Mix hydroxypropylmethylcellulose, glyceryl behenate and mannitol and carefully disperse the dye. Wet with a 5% aqueous solution of polyvinylpyrrolidone. Pass over a 25-mesh sieve, desiccate in an oven (stove) at 30° C. for approximately 2 hours. Pass over the 25-mesh sieve again. Desiccate until constant weight is reached. Add the colloidal silica and magnesium stearate to the granulate obtained and mix in turbula for 15 minutes. In this way a granulate (granulate 13(b)) is obtained with good slide and compaction properties. The granulate is subjected to the compression stage as described later on.

1(c) Preparation of the Three-Layer Systems (by Compression).

The granulates, obtained as previously quoted and according to well known models to all experts in the field, are loaded in the feedboxes of a rotary compressing machine suitable for producing three-layer tablets (e.g. Manesty Layer-Press LP 39, Liverpool, UK). In particular, the granulate described in section 13(b) is loaded in the first and third feedboxes; whereas the granulate as described in section 13(a) is loaded in the second feedbox. The compressing machine is equipped with a slightly concave 9 mm diameter circular punches.

The machine is set so as to produce three-layer systems consisting of an initial 130.0 mg of granulate barrier, a second layer of 150 mg containing ropinirole (0.86 mg of Ropinirole HCl equal to 0.75 mg of base) and a third layer of 120.0 mg of granulate barrier. Working as previously described, three-layer tablets are obtained with a mean weight of 400.00 mg, each containing 0.86 mg of Ropinirole HCl, each equal to 0.75 mg of base. Table 2 contains the data relating to the release verification of the active principle from the tablets in Example 1.

### EXAMPLE 1A

### Variation of Example 1

In an alternative embodiment to Example 1, the preparation of the granulate for the active layer 1(a) is done as follows:

Mix the HPMC, ropinirole, lactose, Na CMC, hydrogenated castor oil and maltodextrin for 6 minutes in a suitable mixer-granulator (type Niro Fielder PMA). Wet with water (approx. 30% of the weight of the products utilised). The granule obtained is desiccated in a fluid bed dryer (type Niro Fielder TSG 2) until the water content is between 1 and 4.5%. Pass through a 1.57 mm mesh sieve in a cone mill. Add silica and mix in a cubic mixer for 20 minutes, then add the magnesium stearate and mix for a further 2 minutes.

In this alternative embodiment, the preparation of the granulate for the barrier layer 1(b) is done as follows:

Mix the mannitol, the dye, the glyceryl behenate, the HPMC, and the PVP for 6 minutes in a suitable mixer-granulator (type Niro Fielder PMA). Wet with water (approx. 25% of the weight of the products utilised). The granule obtained is desiccated in a fluid bed dryer (type Niro Fielder TSG 2) until water content is between 1.1 and 2.7%. Pass through a 1.57 mm mesh sieve in a cone mill. Add silica and mix in a cubic mixer for 20 minutes, then add the magnesium stearate and mix for a further 2 minutes.

In further alternative embodiments, the above alternative procedures can also be used, mutatis mutandis, with the ingredients/formulations of any of the Examples 2 to 11 hereinafter.

### EXAMPLE 2

### Systems Consisting of a Single Three-Layer Tablet—1.00 mg Ropinirole

In Example 2, the first layer consists of 130 mg of a “barrier” layer, the second layer (slow release) contains 1.14 mg of Ropinirole HCl equal to 1.00 mg of base; the third layer consists of 120 mg of a “barrier” layer.

A granulate is prepared as described in Example 1, in section 1(a), the only alteration being to increase the amount of carried active principle, reducing the lactose content by the same amount; such granulate makes up the second layer of the three-layer tablet.

2(a) Preparation of the Granulate Utilised for the Preparation of the Slow Release Layer Containing 1.14 mg of Ropinirole HCl Equal to 1.00 mg of Base.

| Component | Amount (mg) |
|---|---|
| Ropinirole HCl equal to 1.00 mg of base | 1.14 mg |
| Hydroxypropylmethylcellulose (Methocel ® | 61.50 mg |
| K 100 M, Colorcon,Orpington, UK) | |
| Sodium carboxymethylcellulose (Blanose 9 M31XF) | 15.00 mg |
| Maltodextrin NF (Lycatab DSH) | 7.50 mg |
| Lactose (C. Erba, Milan, I) | 47.46 mg |
| Hydrogenated castor oil (Cutina HR-Henkel, D) | 15.00 mg |
| Magnesium stearate (C. Erba, Milan, I) | 1.50 mg |
| Colloidal silica (Syloid 244, Grace GmbH, Worms, D) | 0.90 mg |
| Total | 150.00 mg |

For the first and third layer (barrier) a qualitatively and quantitatively identical granulate is employed, as described in Example 1 in section 1 (b). The compressing machine is equipped with slightly concave 9 mm diameter circular punches.

The machine is set so as to produce three-layer systems consisting of an initial 130.0 mg of granulate barrier, a second layer of 150 mg containing 1.14 mg of Ropinirole HCl (equal to 1.00 mg of Ropinirole base) and a third layer of 120.0 mg of granulate barrier. Working as previously described, three-layer tablets are obtained with a mean weight of 400.00 mg, each containing 1.14 mg of Ropinirole HCl, equal to 1.00 mg of base. Table 2 contains the data relating to the release verification of the active principle from the tablets in Example 2.

### EXAMPLE 3

### Systems Consisting of a Single Three-Layer Tablet—3.00 mg Ropinirole

In Example 3, the first layer consists of 130 mg of a “barrier” layer, the second layer (slow release) contains 3.42 mg of Ropinirole HCl equal to 3.00 mg of base; the third layer consists of 120 mg of “barrier” layer.

A granulate is prepared as described in Example 1, in section 1(a), the only alteration being to increase the amount of carried active principle, reducing the lactose content by the same amount; such granulate makes up the second layer of the three-layer tablet.

3(a) Preparation of the Granulate Utilised for the Preparation of the Slow Release Layer Containing 3.42 mg of Ropinirole HCl Equal to 3.00 mg of Base.

| Component | Amount (mg) |
|---|---|
| Ropinirole HCl equal to 3.00 mg of base | 3.42 mg |
| Hydroxypropylmethylcellulose (Methocel ® K | 61.50 mg |
| 100 M, Colorcon, Orpington, UK) | |
| Sodium carboxymethylcellulose (Blanose 9 M31XF) | 15.00 mg |
| Maltodextrin NF (Lycatab DSH) | 7.50 mg |
| Lactose (C. Erba, Milan, I) | 45.18 mg |
| Hydrogenated castor oil (Cutina HR-Henkel, D) | 15.00 mg |
| Magnesium stearate (C. Erba, Milan, I) | 1.50 mg |
| Colloidal silica (Syloid 244, Grace GmbH, Worms, D) | 0.90 mg |
| Total | 150.00 mg |

For the first and third layer (barrier) a qualitatively and quantitatively identical granulate is employed, as described in Example 1 in section 1(b). The compressing machine is equipped with slightly concave 9 mm diameter circular punches.

The machine is set so as to produce three-layer systems consisting of an initial 130.0 mg of granulate barrier, a second layer of 150 mg containing 3.42 mg of Ropinirole HCl (equal to 3.00 mg of Ropinirole base) and a third layer of 120.0 mg of granulate barrier. Working as previously described, three-layer tablets are obtained with a mean weight of 400.00 mg, each containing 3.42 mg of Ropinirole HCl, each equal to 3.00 mg of base. Table 2 contains the data relating to the release verification of the active principle from the tablets in Example 3.

### EXAMPLE 4

### Systems Consisting of a Single Three-Layer Tablet—6.00 mg Ropinirole

In Example 4, the first layer consists of 130 mg of a “barrier” layer, the second layer (slow release) contains 6.84 mg of Ropinirole HCl equal to 6.00 mg of base; the third layer consists of 120 mg of a “barrier” layer.

A granulate is prepared as described in Example 1, in section 1(a), the only alteration being to increase the amount of carried active principle, reducing the lactose content by the same amount; such granulate makes up the second layer of the three-layer tablet.

4(a) Preparation of the Granulate Utilised for the Preparation of the Slow Release Layer Containing 6.84 mg of Ropinirole HCl Equal to 6.00 mg of Base.

| Component | Amount (mg) |
|---|---|
| Ropinirole HCl equal to 6.00 mg of base | 6.84 mg |
| Hydroxypropylmethylcellulose (Methocel ® K | 61.50 mg |
| 100 M, Colorcon, Orpington, UK) | |
| Sodium carboxymethylcellulose (Blanose 9 M31XF) | 15.00 mg |
| Maltodextrin NF (Lycatab DSH) | 7.50 mg |
| Lactose (C. Erba, Milan, I) | 41.76 mg |
| Hydrogenated castor oil (Cutina HR-Henkel, D) | 15.00 mg |
| Magnesium stearate (C. Erba, Milan, I) | 1.50 mg |
| Colloidal silica (Syloid 244, Grace GmbH, Worms, D) | 0.90 mg |
| Total | 150.00 mg |

For the first and third layer (barrier) a qualitatively and quantitatively identical granulate is employed, as described in Example 1 in section 1(b). The compressing machine is equipped with slightly concave 9 mm diameter circular punches. The machine is set so as to produce three-layer systems consisting of an initial 130.0 mg of granulate barrier, a second layer of 150 mg containing 6.84 mg of Ropinirole HCl (equal to 6.00 mg of Ropinirole base) and a third layer of 120.0 mg of granulate barrier. Working as previously described, three-layer tablets are obtained with a mean weight of 400.00 mg, each containing 6.84 mg of Ropinirole HCl, equal to 6.00 mg of base. Table 2 contains the data relating to the release verification of the active principle from the tablets in Example 4.

### EXAMPLE 5

### Systems Consisting of a Single Three-Layer Tablet—9.00 mg Ropinirole

In Example 5, the first layer consists of 130 mg of a “barrier” layer, the second layer (slow release) contains 10.26 mg of Ropinirole HCl equal to 9.00 mg of base; the third layer consists of 120 mg of a “barrier” layer.

A granulate is prepared as described in Example 1, in section 1(a), the only alteration being to increase the amount of carried active principle, reducing the lactose content by the same amount; such granulate makes up the second layer of the three-layer tablet.

5(a) Preparation of the granulate utilised for the preparation of the slow release layer containing 10.26 mg of Ropinirole HCl equal to 9.00 mg of base.

| Component | Amount (mg) |
|---|---|
| Ropinirole HCl equal to 9.00 mg of base | 10.26 mg |
| Hydroxypropylmethylcellulose (Methocel ® K | 61.50 mg |
| 100 M, Colorcon, Orpington, UK) | |
| Sodium carboxymethylcellulose (Blanose 9 M31XF) | 15.00 mg |
| Maltodextrin NF (Lycatab DSH) | 7.50 mg |
| Lactose (C. Erba, Milan, I) | 38.34 mg |
| Hydrogenated castor oil (Cutina HR-Henkel, D) | 15.00 mg |
| Magnesium stearate (C. Erba, Milan, I) | 1.50 mg |
| Colloidal silica (Syloid 244, Grace GmbH, Worms, D) | 0.90 mg |
| Total | 150.00 mg |

For the first and third layer (barrier) a qualitatively and quantitatively identical granulate is employed, as described in Example 1 in section 1(b). The compressing machine is equipped with 8 mm diameter circular concave punches. The machine is set so as to produce three-layer systems consisting of an initial 130.0 mg of granulate barrier, a second layer of 150 mg containing the active principle (equal to 9.00 mg of Ropinirole base) and a third layer of 120.0 mg of granulate barrier. Working as previously described, three-layer tablets are obtained with a mean weight of 400.00 mg, each containing 9.00 mg of active principle. Table 2 contains the data relating to the release verification of the active principle from the tablets in Example 5.

### EXAMPLE 6

### Systems Consisting of a Single Three-Layer Tablet—12.00 mg Ropinirole

In Example 6, the first layer consists of 130 mg of a “barrier” layer, the second layer (slow release) contains 13.68 mg of Ropinirole HCl equal to 12.00 mg of base; the third layer consists of 120 mg of a “barrier” layer.

A granulate is prepared as described in Example 1, in section 1(a), the only alteration being to increase the amount of carried active principle, reducing the lactose content by the same amount; such granulate makes up the second layer of the three-layer tablet.

6(a) Preparation of the Granulate Utilised for the Preparation of the Slow Release Layer Containing 13.68 mg of Ropinirole HCl equal to 12.00 mg of Base.

| Component | Amount (mg) |
|---|---|
| Ropinirole HCl equal to 12.00 mg of base | 13.68 mg |
| Hydroxypropylmethylcellulose (Methocel ® K | 61.50 mg |
| 100 M, Colorcon, Orpington, UK) | |
| Sodium carboxymethylcellulose (Blanose 9 M31XF) | 15.00 mg |
| Maltodextrin NF (Lycatab DSH) | 7.50 mg |
| Lactose (C. Erba, Milan, I) | 34.92 mg |
| Hydrogenated castor oil (Cutina HR-Henkel, D) | 15.00 mg |
| Magnesium stearate (C. Erba, Milan, I) | 1.50 mg |
| Colloidal silica (Syloid 244, Grace GmbH, Worms, D) | 0.90 mg |
| Total | 150.00 mg |

For the first and third layer (barrier) a qualitatively and quantitatively identical granulate is employed, as described in Example 1 in section 1(b). The compressing machine is equipped with 8 mm diameter circular concave punches. The machine is set so as to produce three-layer systems consisting of an initial 130.0 mg of granulate barrier, a second layer of 150 mg containing the active principle (equal to 12.00 mg of Ropinirole base) and a third layer of 120.0 mg of granulate barrier. Working as described previously, three-layer tablets are obtained with a mean weight of 400.00 mg, each containing 12.00 mg of the active principle. Table 2 contains the data relating to the release verification of the active principle from the tablets in Example 18.

Dissolution Test of Tablets Prepared in Examples 1 to 6

To assess the release characteristics of the active principle from the three-layer tablets, quoted in Examples 1-6, equipment 2 is utilised, paddle (USP XXIII), working at 100 r.p.m. and utilising as dissolution fluid 500 mL of aqueous buffer solution of citrate (pH4.0), at 37° C. The release of the active principle is followed by HPLC assessment at 250 nm utilising an automatic sampling and reading system. The results of the experiments carried out are quoted in Table 2.

TABLE 2

| | |
|---|---|
| | Percentage Released in Tablets |
| TIME | of Examples 1 to 6 (Ex. 1 to Ex. 6) |

| (hours) | Ex. 1 | Ex. 2 | Ex. 3 | Ex. 4 | Ex. 5 | Ex. 6 |
|---|---|---|---|---|---|---|
| 1 | 7.3 | 8.6 | 7.8 | 7.5 | 8.8 | 9.4 |
| 2 | 12.1 | 12.6 | 12.0 | 13.4 | 13.5 | 14.0 |
| 4 | 18.9 | 21.0 | 19.5 | 20.7 | 22.1 | 23.9 |
| 6 | 26.0 | 28.5 | 27.9 | 28.5 | 29.8 | 33.1 |
| 9 | 38.3 | 39.7 | 39.2 | 40.3 | 41.2 | 44.9 |
| 12 | 49.6 | 51.4 | 50.7 | 51.0 | 52.6 | 56.7 |
| 16 | 67.8 | 66.9 | 64.5 | 66.3 | 66.4 | 70.0 |
| 20 | 82.0 | 81.3 | 78.4 | 79.5 | 80.3 | 80.1 |
| 24 | 90.4 | 91.3 | 88.9 | 89.1 | 88.7 | 91.2 |

It is possible to point out that the release of the drug from the prepared systems is slowed down and the release of most of the drug takes approximately 24 hours.

In addition, it is clear that the release kinetics are not substantially modified at any content of active substance in the tablets. Such behaviour is in accordance with the present invention.

### EXAMPLE 7

### Preparation of Ropinirole Round Tablet Formula

Tablet formulations of ropinirole as a round tablet were prepared as follows. The tablet comprised an upper support or barrier layer (1), an active layer (2) and a lower support or barrier layer (3). HPMC is an abbreviation for hydroxypropylmethylcellulose.

Support Layer (1)

| | | Amount |
|---|---|---|
| Component | Role | mg/tablet |
| HPMC type 2208/K 100 M | Hydrophilic matrix polymer | 58.18 |
| (100,000 cps) | | |
| Mannitol | Filler, Diluent | 30.68 |
| Glyceryl behenate | Hydrophobic compound | 32.50 |
| Polyvinylpyrrolidone (Povidone) | Binder | 6.50 |
| Magnesium stearate | Lubricant | 1.30 |
| Colloidal silicon dioxide | Glidant | 0.52 |
| Yellow ferric oxide | Colouring agent | 0.33 |
| Purified water | Granulation liquid | b |
| Total | | 130.00 |

Active Layer (2)
Described in terms of three tablet strength formulations
of ropinirole at 0.75 mg, 1 mg or 3 mg per tablet
ropinirole measured as effective base present.

| | | Amount mg/tablet | | |
|---|---|---|---|---|
| Component | Role | 0.75 mg | 1 mg | 3 mg |
| Ropinirole HCl | Active Substance | 0.855 | 1.14 | 3.42 |
| Lactose monohydrate | Filler, diluent | 47.745 | 47.46 | 45.18 |
| HPMC type 2208/K | Hydrophobic matrix | 61.50 | 61.50 | 61.50 |
| 100 M (100,000 cps) | polymer | (41 wt %) | | |
| Carboxymethylcellulose sodium | Viscosity regulating agent | 15.00 | 15.00 | 15.00 |
| Maltodextrin | Binder | 7.50 | 7.50 | 7.50 |
| Hydrogenated castor oil | Hydrophobic compound | 15.00 | 15.00 | 15.00 |
| Magnesium stearate | Lubricant | 1.50 | 1.50 | 1.50 |
| colloidal silicon dioxide | Glidant | 0.90 | 0.90 | 0.90 |
| Purified water | Granulation liquid | b | b | b |
| Total | | 150.00 | 150.00 | 150.00 |

Support Layer (3)

| | | Amount |
|---|---|---|
| Component | Role | mg/tablet |
| HPMC type 2208/K 100 M | Hydrophilic matrix polymer | 53.70 |
| (100,000 cps) | | |
| Mannitol | Filler, Diluent | 28.32 |
| Glyceryl behenate | Hydrophobic compound | 30.00 |
| Polyvinylpyrrolidone (Povidone) | Binder | 6.00 |
| Magnesium stearate | Lubricant | 1.20 |
| Colloidal silicon dioxide | Glidant | 0.48 |
| Yellow ferric oxide | Colouring agent | 0.30 |
| Purified water | Granulation liquid | b |
| Total | | 120.00 |

The purified water included as granulation liquid does not remain in the finished product as indicated by the reference sign “b”.

### EXAMPLE 8

### Preparation of Ropinirole Caplet Formula

Tablet formulations of ropinirole as a caplet were prepared as follows. The tablet comprised an upper support or barrier layer (1), an active layer (2) and a lower support or barrier layer (3), as shown in FIG. 9 (in which reference numerals 10,12 represent the barrier layers and 11 the active layer). HPMC is an abbreviation for hydroxypropylmethylcellulose. Described in terms of four tablet strength formulations of ropinirole at 1 mg, 3 mg, 6 mg, 9 mg or 12 mg per tablet ropinirole measured as effective base present.

Support layer (1)

| | | Amount ropinirole (mg/tablet) | | | |
|---|---|---|---|---|---|
| Component | Role | 1 mg | 3 mg | 6 or 9 mg | 12 mg |
| HPMC type 2208 | Hydrophilic matrix | 76.07 | 76.07 | 76.07 | 76.07 |
| (100,000 cps) | polymer | | | | |
| Mannitol | Filler, diluent | 40.12 | 40.12 | 40.12 | 40.12 |
| Glyceryl behenate | Hydrophobic compound | 42.50 | 42.50 | 42.50 | 42.50 |
| Polyvinylpyrrolidone | Binder | 8.50 | 8.50 | 8.50 | 8.50 |
| (Povidone) | | | | | |
| Magnesium stearate | Lubricant | 1.70 | 1.70 | 1.70 | 1.70 |
| Colloidal silicon dioxide | Glidant | 0.68 | 0.68 | 0.68 | 0.68 |
| Yellow ferric oxide | Colouring agent | 0.43 | 0.43 | 0.43 | 0.43 |
| Purified water | Granulation liquid | c | c | c | c |
| Total | | 170.00 | 170.00 | 170.00 | 170.00 |

Active layer (2)

| | | Amount ropinirole (mg/tablet) | | | | |
|---|---|---|---|---|---|---|
| Component | Role | 1 mg | 3 mg | 6 mg | 9 mg | 12 mg |
| Ropinirole HCl | Active substance | 1.14 | 3.42 | 6.84 | 10.26 | 13.68 |
| Lactose monohydrate | Filler, diluent | 47.46 | 45.18 | 41.76 | 38.34 | 34.92 |
| HPMC type 2208 | Hydrophilic matrix | 61.50 | 61.50 | 61.50 | 61.50 | 61.50 |
| (100,000 cps) | polymer | (41 | | | | |
| | | wt %) | | | | |
| Carboxymethyl- | Viscosity regulating | 15.00 | 15.00 | 15.00 | 15.00 | 15.00 |
| cellulose sodium | agent | | | | | |
| Maltodextrin | Binder | 7.50 | 7.50 | 7.50 | 7.50 | 7.50 |
| Hydrogenated castor | Hydrophobic | 15.00 | 15.00 | 15.00 | 15.00 | 15.00 |
| oil | compound | | | | | |
| Magnesium stearate | Lubricant | 1.50 | 1.50 | 1.50 | 1.50 | 1.50 |
| Colloidal silicon | Glidant | 0.90 | 0.900 | 0.90 | 0.90 | 0.90 |
| dioxide | | | | | | |
| Purified water | Granulation liquid | c | c | c | c | c |
| Total | | 150.00 | 150.00 | 150.00 | 150.00 | 150.00 |

Support layer (3)

| | | Amount ropinirole (mg/tablet) | | | |
|---|---|---|---|---|---|
| Component | Role | 1 mg | 3 mg | 6 or 9 mg | 12 mg |
| HPMC type 2208 | Hydrophilic matrix | 62.65 | 62.65 | 62.65 | 62.65 |
| (100,000 cps) | polymer | | | | |
| Mannitol | Filler, diluent | 33.04 | 33.04 | 33.04 | 33.04 |
| Glyceryl behenate | Hydrophobic compound | 35.00 | 35.00 | 35.00 | 35.00 |
| Polyvinylpyrrolidone | Binder | 7.00 | 7.00 | 7.00 | 7.00 |
| (Povidone) | | | | | |
| Magnesium stearate | Lubricant | 1.40 | 1.40 | 1.40 | 1.40 |
| Colloidal silicon dioxide | Glidant | 0.56 | 0.56 | 0.56 | 0.56 |
| Yellow ferric oxide | Colouring agent | 0.35 | 0.35 | 0.35 | 0.35 |
| Purified water | Granulation liquid | c | c | c | c |
| Total | | 140.00 | 140.00 | 140.00 | 140.00 |

Film coating

| | | Amount ropinirole (mg/tablet) | | | |
|---|---|---|---|---|---|
| | | | | 6 or | |
| Component | Role | 1 mg | 3 mg | 9 mg | 12 mg |
| OPADRY OY-S- | Coating agent | 13.80 | 13.80 | 13.80 | 13.80 |
| 28876 WHITE | | | | | |
| Purified water | Coating liquid | c | c | c | c |
| Total tablet | | 473.80 | 473.80 | 473.80 | 473.80 |
| weight (layers 1, | | | | | |
| 2, 3 and coat) | | | | | |

The purified water included as granulation liquid or coating liquid does not remain in the finished product as indicated by the reference sign “c”.OPADRY OY-S-28876 WHITE is 63% HPMC 2910 6cP, 7% PEG 400, 30% TiO2. Red/pink (0.01-0.25%) and/or yellow (0.1 to 1.5%) colourings can also be added (iron oxides), the HPMC varying between 61-66%. An alternative blue coating uses 31-32% each of HPMC 2910 3cP and HPMC 2910 5cP, 8% PEG400, 23-24% TiO2, 1% polysorbate, and indigotine as blue dye at 4-5%.

### EXAMPLE 9

### Preparation of Ropinirole Caplet Formula

Tablet formulations of ropinirole as a caplet were prepared as follows. The tablet comprised an upper support or barrier layer (1), an active layer (2) and a lower support or barrier layer (3), as with Example 8. Described in terms of four tablet strength formulations of ropinirole at 1 mg, 3 mg, 6 mg, 9 mg and 12 mg per tablet ropinirole measured as effective base present. This example is the same as Example 8 but the yellow ferric oxide in the support or barrier layers are absent.

Support layer (1)

| | | Amount |
|---|---|---|
| | | ropinirole |
| | | (mg/tablet) |
| Component | Role | 1, 3 or 6 mg |
| HPMC type 2208 (100,000 cps) | Hydrophilic matrix | 76.50 |
| | polymer | |
| Mannitol | Filler, diluent | 40.12 |
| Glyceryl behenate | Hydrophobic compound | 42.50 |
| Polyvinylpyrrolidone (Povidone) | Binder | 8.50 |
| Magnesium stearate | Lubricant | 1.70 |
| Colloidal silicon dioxide | Glidant | 0.68 |
| Purified water | Granulation liquid | d |
| Total | | 170.00 |

Active layer (2)

| | | Amount ropinirole (mg/tablet) | | | | |
|---|---|---|---|---|---|---|
| Component | Role | 1 mg | 3 mg | 6 mg | 9 mg | 12 mg |
| Ropinirole HCl | Active substance | 1.14 | 3.42 | 6.84 | 10.26 | 13.68 |
| Lactose | Filler, diluent | 47.46 | 45.18 | 41.76 | 38.34 | 34.92 |
| monohydrate | | | | | | |
| HPMC type 2208 | Hydrophilic matrix | 61.50 | 61.50 | 61.50 | 61.50 | 61.50 |
| (100,000 cps) | polymer | (41 | | | | |
| | | wt %) | | | | |
| Carboxymethyl- | Viscosity regulating | 15.00 | 15.00 | 15.00 | 15.00 | 15.00 |
| cellulose sodium | agent | | | | | |
| Maltodextrin | Binder | 7.50 | 7.50 | 7.50 | 7.50 | 7.50 |
| Hydrogenated castor | Hydrophobic | 15.00 | 15.00 | 15.00 | 15.00 | 15.00 |
| oil | compound | | | | | |
| Magnesium stearate | Lubricant | 1.50 | 1.50 | 1.50 | 1.50 | 1.50 |
| Colloidal silicon | Glidant | 0.90 | 0.900 | 0.90 | 0.90 | 0.90 |
| dioxide | | | | | | |
| Purified water | Granulation liquid | c | c | c | c | c |
| Total | | 150.00 | 150.00 | 150.00 | 150.00 | 150.00 |

Support layer (3)

| | | Amount |
|---|---|---|
| | | ropinirole |
| | | (mg/tablet) |
| Component | Role | 1, 3 or 6 mg |
| HPMC type 2208 (100,000 cps) | Hydrophilic matrix | 63.00 |
| | polymer | |
| Mannitol | Filler, diluent | 33.04 |
| Glyceryl behenate | Hydrophobic compound | 35.00 |
| Polyvinylpyrrolidone | Binder | 7.00 |
| (Povidone) | | |
| Magnesium stearate | Lubricant | 1.40 |
| Colloidal silicon dioxide | Glidant | 0.56 |
| Purified water | Granulation liquid | d |
| Total | | 140.00 |

Film coating

| | | Amount ropinirole (mg/tablet) | | | |
|---|---|---|---|---|---|
| Component | Role | 1 mg | 3 mg | 6 or 9 mg | 12 mg |
| OPADRY OY- | Coating agent | 13.80 | 13.80 | 13.80 | 13.80 |
| S-28876 | | | | | |
| WHITE | | | | | |
| Purified water | Coating liquid | d | d | d | d |
| Total tablet | | 473.80 | 473.80 | 473.80 | 473.80 |
| weight (layers | | | | | |
| 1, 2, 3 and | | | | | |
| coat) | | | | | |

The purified water included as granulation liquid or coating liquid does not remail in the finished product as indicated by the reference sign “d”.

The purified water included as granulation liquid or coating liquid does not remain in the finished product as indicated by the reference sign “d”.

### EXAMPLES 10 AND 11

### Preparation of Ropinirole Caplet Formula

Tablet formulations of ropinirole as a caplet were prepared as follows. The tablet comprised an upper support or barrier layer (1), an active layer (2) and a lower support or barrier layer (3), as for Examples 8 and 9. Example formulations 10 and 11 are described as a single tablet strength formulation each of ropinirole at 0.75 mg per tablet ropinirole measured as effective base present (0.855 mg measured as the HCl salt). As can be seen, Examples 10 and 11 involve identical active layers to the 0.75 mg ropinirole active layer of Example 7, but different barrier layers to Example 7, with different amounts and grades of HPMC in the barrier layers, replacement of mannitol with lactose, and lower amounts of glyceryl behenate. As can be seen, Examples 10 and 11 have 10% and 40 weight % respectively of K4M HPMC in their barrier layers which gives a slightly faster release profile in vitro than the ca. 45 wt % K100M HPMC present in the barrier layers of Examples 1-6 and 7-9, as inter alia the ropinirole migrates faster through the barrier layers. Example 10 has 20 wt % of K100LV HPMC as well as 10 wt % K4M HPMC in the barrier layer—the low-viscosity (LV) HPMC in the barrier layer may increase water uptake and aid gelling, increasing the matrix viscosity and decreasing the release rate.

Support layer (1) for Examples 10, 11

| | | Amount (mg/tablet) | |
|---|---|---|---|
| | | Exam- | Ex- |
| Component | Role | ple 10 | ample 11 |
| HPMC K 4M | Hydrophilic matrix | 13.00 | 51.84 |
| | polymer | | |
| HPMC K100LV | Hydrophilic matrix | 26.00 | |
| | polymer | | |
| Lactose monohydrate | | 64.68 | 51.84 |
| Glyceryl behenate | Hydrophobic compound | 17.56 | 17.55 |
| Polyvinylpyrrolidone | Binder | 6.50 | 6.50 |
| (Povidone) | | | |
| Magnesium stearate | Lubricant | 1.30 | 1.30 |
| Colloidal silicon dioxide | Glidant | 0.64 | 0.63 |
| Yellow ferric oxide | Colouring agent | 0.32 | 0.32 |
| Purified water | Granulation liquid | c | c |
| Total | | 130.00 | 130.00 |

Active Layer (2) for Examples 10 and 11
Described in terms of three tablet strength formulations
of ropinirole at 0.75 mg per tablet ropinirole measured
as effective base present.

| | | Amount ropinirole |
|---|---|---|
| | | mg/tablet |
| Component | Role | 0.75 mg |
| Ropinirole HCl | Active Substance | 0.855 |
| Lactose monohydrate | Filler, diluent | 47.745 |
| HPMC type 2208/K 100M | Hydrophobic matrix | 61.50 |
| (100,000 cps) | polymer | |
| Carboxymethylcellulose | Viscosity regulating | 15.00 |
| sodium | agent | |
| Maltodextrin | Binder | 7.50 |
| Hydrogenated castor oil | Hydrophobic | 15.00 |
| | compound | |
| Magnesium stearate | Lubricant | 1.50 |
| Colloidal silicon dioxide | Glidant | 0.90 |
| Purified water | Granulation liquid | c |
| Total | | 150.00 |

Support layer (3) for Examples 10, 11

| | | Amount | |
|---|---|---|---|
| | | (mg/tablet) | |
| | | Exam- | Exam- |
| Component | Role | ple 10 | ple 11 |
| HPMC K4M | Hydrophilic matrix | 12.00 | 47.86 |
| | polymer | | |
| HPMC K100LV | | 24.00 | |
| Lactose monohydrate | | 59.70 | 47.86 |
| Glyceryl behenate | Hydrophobic compound | 16.20 | 16.20 |
| Polyvinylpyrrolidone | Binder | 6.00 | 6.00 |
| (Povidone) | | | |
| Magnesium stearate | Lubricant | 1.20 | 1.20 |
| Colloidal silicon dioxide | Glidant | 0.60 | 0.60 |
| Yellow ferric oxide | Colouring agent | 0.30 | 0.30 |
| Purified water | Granulation liquid | c | c |
| Total | | 120.00 | 120.00 |

The purified water included as granulation liquid or coating liquid does not remain in the finished product as indicated by the reference sign “c”.

Note: The active layer for each Example 10 and 11 above can be replaced by the active layers of Examples 8 and 9 using 1 mg, 3 mg, 6 mg, 9 mg and 12 mg ropinirole measured as effective base present.

Note: in all the ropinirole Examples 1-6 and 7-11, higher doses of up to 24 mg ropinirole per day can be administered by e.g. 2×12 mg tablets. Other doses e.g. 4 mg per day can be administered using 1×1 mg and 1×3 mg tablet per day. Also, in any of the Examples 1-11, different doses of for example 0.25 mg, 0.5 mg and 2 mg ropinirole can be used in the active layer by varying the amount of lactose while keeping the total weight of the active layer constant.

### EXAMPLE 12

### Further Studies on Caplet Preparation and Drug Dissolution Profiles

The following further studies on caplet preparation are presented to show drug dissolution profiles for ropinirole caplets containing 0.75 mg, 6 mg or 12 mg ropinirole measured as effective base present.

Formulation of Ropinirole HCl CR caplet, detailed per layer, mg/tablet
Ingredients

| Layer 1, support layer D14-4 Yellow | | | |
|---|---|---|---|
| HPMC type 2208 (100 000 cps) | 76.075 | | |
| Mannitol | 40.120 | | |
| Glyceryl behenate | 42.500 | | |
| Povidone | 8.500 | | |
| Yellow ferric oxide | 0.425 | | |
| Magnesium stearate vegetable | 1.700 | | |
| Colloidal silicon dioxide | 0.680 | | |
| Purified water | a | | |
| Total | 170.000 | | |
| Layer 2, active layer | 0.75 mg | 6 mg | 12 mg |
| Ropinirole HCl | 0.855 | 6.840 | 13.680 |
| Lactose monohydrate | 47.745 | 41.760 | 34.920 |
| HPMC type 2208 (100 000 cps) | 61.500 | 61.500 | 61.500 |
| Carboxymethylcellulose sodium | 15.000 | 15.000 | 15.000 |
| Maltodextrin | 7.500 | 7.500 | 7.500 |
| Hydrogenated castor oil | 15.000 | 15.000 | 15.000 |
| Magnesium stearate vegetable | 1.500 | 1.500 | 1.500 |
| Colloidal silicon dioxide | 0.900 | 0.900 | 0.900 |
| Purified water | a | a | a |
| Total, layer 2 | 150.000 | 150.000 | 150.000 |

| | |
|---|---|
| Layer 3, support layer D14-4 Yellow | |
| HPMC type 2208 (100 000 cps) | 62.650 |
| Mannitol | 33.040 |
| Glyceryl behenate | 35.000 |
| Povidone | 7.000 |
| Yellow ferric oxide | 0.350 |
| Magnesium stearate vegetable | 1.400 |
| Colloidal silicon dioxide | 0.560 |
| Purified water | a |
| Total | 140.000 |
| Film coating | |
| OPADRY OY-S-28876 WHITE | 13.800 |
| Purified water | a |
| Total, tablet | 473.800 |

Notes:
a = Does not remain in the final product
0.855 mg of Ropinirole HCl is corresponding to 0.75 mg of Ropinirole Base
6.840 mg of Ropinirole HCl is corresponding to 6.00 mg of Ropinirole Base
13.680 mg of Ropinirole HCl is corresponding to 12.00 mg of Ropinirole Base

The drug release profiles were measured using standard techniques. The results were as follows (results presented in terms of percentage drug released at given time intervals in hours):

Analytical results

| | Dosage, mg | | | | | | | | |
|---|---|---|---|---|---|---|---|---|---|
| | 0.75 | 0.75 | 0.75 | 6 | 6 | 6 | 12 | 12 | 12 |
| Batch no. | C511 | C519 | C529 | C530 | C531 | C532 | C512 | C534 | C535 |
| Tablet | P00K41E | P00K40E | P00K39E | P00K45E | P00K46E | P00K47E | P00K42E | P00K43E | P00K44E |
| Tablet weight, mg | 471.95 | 472.32 | 472.08 | 474.08 | 471.50 | 473.37 | 470.39 | 473.62 | 474.78 |
| Tablet wt RSD, % | 0.78 | 1.44 | 0.65 | 0.98 | 1.10 | 1.08 | 0.93 | 1.28 | 1.02 |
| Time, h | | | | | | | | | |
| 0.00 | 0.00 | 0.00 | 0.00 | 0.00 | 0.00 | 0.00 | 0.00 | 0.00 | 0.00 |
| 1.00 | 8.96 | 6.88 | 7.43 | 8.67 | 8.20 | 8.71 | 9.45 | 10.10 | 9.73 |
| 2.00 | 12.42 | 11.45 | 11.69 | 13.47 | 13.17 | 13.48 | 14.87 | 15.53 | 15.23 |
| 4.00 | 19.99 | 19.93 | 19.59 | 22.03 | 22.01 | 21.55 | 24.37 | 24.87 | 24.55 |
| 6.00 | 27.45 | 27.62 | 27.43 | 30.21 | 29.65 | 30.17 | 33.38 | 33.74 | 33.33 |
| 9.00 | 38.24 | 38.60 | 38.34 | 41.68 | 41.46 | 41.81 | 45.56 | 46.22 | 45.81 |
| 12.00 | 49.78 | 49.58 | 50.00 | 52.07 | 52.36 | 52.33 | 56.81 | 57.40 | 56.71 |
| 16.00 | 64.53 | 64.48 | 65.47 | 66.17 | 66.41 | 66.26 | 69.54 | 70.90 | 69.52 |
| 20.00 | 77.17 | 76.98 | 78.68 | 78.01 | 78.34 | 78.72 | 80.95 | 81.64 | 79.95 |
| 24.00 | 85.79 | 86.17 | 88.18 | 87.09 | 87.69 | 88.19 | 89.07 | 89.76 | 88.12 |
| 30.00 | 92.40 | 93.57 | 95.40 | 95.26 | 94.81 | 95.41 | 95.76 | 96.63 | 94.60 |
| 36.00 | 94.37 | 96.00 | 97.19 | 97.96 | 97.17 | 97.58 | 97.80 | 99.38 | 97.26 |

The contents of international patent application PCT/GB01/01726 are herein incorporated by reference.

1-29. Cancelled.

30. A method for treating fibromyalgia comprising administering a multi-layer controlled-release tablet to human in need thereof wherein said multi-layer controlled-release tablet comprises:
(a) one active layer comprising:
(i) ropinirole or a pharmaceutically acceptable salt thereof,
(ii) two or more hydrophilic polymeric substances,
(iii) one or more lipophilic substance, and
(iv) one ore more optional adjuvant substance,
wherein the weight ratio of the one ore more hydrophilic polymeric substance to the one or more lipophilic substance contained in said active layer is from aout 10:1 to about 0.5:1; and
(b) one or more barrier layer.

31. The method according to claim 30, wherein said one or more barrier layer comprises:
(a) two or more hydrophilic polymeric substances,
(b) one or more lipophilic substance, and
(c) one ore more optional adjuvant substance.

32. The method according to claim 30, wherein the weight ratio of the two or more hydrophilic polymeric substances to the one or more lipophilic substance contained in said active layer is in the range of 7:1 to 1:1.

33. The method according to claim 30, wherein said active layer comprises from 1% to 75% by weight of the two or more hydrophilic polymeric substances.

34. The method according to claim 30, wherein said active layer comprises from 1% to 70% by weight of the one or more lipophilic substance.

35. The method according to claim 30, wherein said active layer comprises from 5% to 50% by weight of the one or more adjuvant substance.

36. The method according to claim 30, wherein the weight ratio between the ropinirole or a pharmaceutically acceptable salt thereof and the one or more adjuvant substance in said active layer is from 0.001:1 to 4:1.

37. The method according to claim 30, wherein said one or more barrier layer is applied to one or both surfaces of said active layer.

38. The method according to claim 30, wherein the weight ratio of the two or more hydrophilic polymeric substances to the one or more lipophilic substance in said one or more barrier layer is from 1:1 to 7.5:1.

39. The method according to claim 30, wherein said active layer comprises from 0.01% to 70% by weight of the ropinirole or a pharmaceutically acceptable salt thereof.

40. The method according to claim 30, wherein said active layer comprises from 0.05% to 20% by weight of the ropinirole or a pharmaceutically acceptable salt thereof.

41. The method according to claim 30, wherein said active layer further comprises one or more lipophilic polymeric substance.

42. The method according to claim 30, wherein at least one of the two or more hydrophilic polymeric substances is a pharmaceutically acceptable biocompatible and/or biodegradable material selected from the roup consisting of: non-cross-linked polyvinylpyrrolidone, hydroxypropylcellulose with a molecular weight of 100,000 to 4,000,000, sodium carboxymethylcellulose, carboxymethylstarch, potassium methacrylate-divinylbenzene copolymer, hydroxypropylmethylcellulose of molecular weight between 2,000 and 4,000,000, a polyethyleneglycol of molecular weight between 200 and 15,000, a polyoxyethylene of molecular weight up to 20,000,000, a carboxyvinylpolymer, a poloxamer, a polyvinylalcohol, a glucane, a carrageenan, a scleroglucane, a mannan, a galactomannan, gellans, xanthans, alginic acid or a derivative thereof, polyaminoacids, methyl vinyl ether/maleic anhydride copolymer, carboxymethylcellulose or a derivative thereof, ethylcellulose, methylcellulose, starch or a derivative thereof, a dextrin derivative, and alpha, beta, or gamma cyclodextrin.

43. The method according to claim 30, wherein the one or more lipophilic substance is selected from the group consisting of: a natural fat, a totally or partially hydrogenated fat, beeswax, polyethoxylated beeswax, a mono-, bi- or tri-substituted glyceride, glyceryl palmitostearate, glyceryl behenate, diethyleneglycol palmitostearate, a polyethyleneglycol stearate, a polyoxyethyleneglycol palmitostearate, glyceryl monopalmitostearate, cetyl palmitate, polyethyleneglycol palmitostearate, mono- or di-glyceryl behenate, a fatty alcohol associated with a polyethoxylate fatty alcohol, cetyl alcohol, stearic acid, a saturated or unsaturated fatty acid or a hydrogenated derivative thereof, and hydrogenated castor oil.

44. The method according to claim 30, wherein the tablet compression is carried out by compression of the powder or granular mixture between 1000 and 5000 Kg/cm².

45. The method according to claim 30, wherein at least one of the one or more lipophilic substance in said active layer is selected from the group consisting of: hydrogenated castor oil and glyceryl behenate.

46. The method according to claim 30, wherein said active layer contains up to 12.0 mg of ropinirole or a pharmaceutically acceptable salt thereof measured as the amount of ropinirole base present.

47. The method according to claim 46, wherein said active layer contains from 0.75 mg to 12.0 mg of ropinirole or a pharmaceutically acceptable salt thereof measured as the amount of ropinirole base present.

48. The method according to claim 30, wherein said active layer comprises from 0.05% to 20% by weight of ropinirole or a pharmaceutically acceptable salt thereof, from 5% to 50% by weight of the one or more adjuvant substance, and wherein the weight ratio of the two or more hydrophilic polymeric substances to the one or more lipophilic substance contained in said active layer is from 7:1 to 1:1.

49. The method according to claim 31, wherein the two or more hydrophilic polymeric substances are selected from the group consisting of: hydroxypropylmethylcellulose of a molecular weight from 2,000 to 4,000,000, sodium carboxymethylcellulose and calcium carboxymethylcellulose.

50. The method according to claim 40, wherein the two or more hydrophilic polymeric substances are selected from the group consisting of: hydroxypropylmethylcellulose of a molecular weight from 2,000 to 4,000,000, sodium carboxymethylcellulose, and calcium carboxymethylcellulose.

51. The method according to claim 50, wherein at least one of the one or more lipophilic substance is hydrogenated castor oil or glyceryl behenate, wherein said active layer comprises from 0.05% to 20% by weight of ropinirole or a pharmaceutically acceptable salt thereof, from 5% to 50% by weight of the one or more adjuvant substance, and wherein the weight ratio of the two or more hydrophilic polymeric substances to the one or more lipophilic substance contained in said active layer is from 7:1 to 1:1.

52. The method according to claim 51, wherein the two or more hydrophilic polymeric substances are selected from the group consisting of: hydroxypropylmethylcellulose of a molecular weight from 2,000 to 4,000,000 and sodium carboxymethylcellulose.
